(19) United States
(12) Patent Application Publication
(10) Pub. No.: US 2007/0036761 A1
(43) Pub. Date: Feb. 15, 2007

(54) Lentiviral apoe2 gene therapy

(76) Inventors: Kelly Renee Bales, Coatesville, IN (US); Jean-Cosme Francois Dodart, Indianapolis, IN (US); Steven Marc Paul, Carmel, IN (US); Robert A. Marr, San Diego, CA (US); Inder Mohan Verma, San Diego, CA (US)

Correspondence Address: ELI LILLY & COMPANY, PATENT DIVISION, P.O. BOX 6288, INDIANAPOLIS, IN, 46206-6288, US

(21) Appl. No.: 10/595,151

(22) PCT Filed: Sep. 24, 2004
(86) PCT No.: PCT/US04/28811

§ 371 Date: Mar. 7, 2006

Related U.S. Application Data
(60) Provisional application No. 60/506,559, filed on Sep. 26, 2003.

(51) Int. Cl. A61K 48/00; C12N 15/867

(52) U.S. Cl. 424/93.2; 435/456

(57) ABSTRACT

The present invention is a method for inhibiting or reducing disease progression in subjects suffering from conditions or diseases related to the Aβ peptide, including Alzheimer's disease, Down's syndrome, cerebral amyloid angiopathy, mild cognitive impairment, and the like. The method comprises admininstering an apoE2 lentiviral vector to the subject.

### BACKGROUND OF THE INVENTION

The invention relates to lentivirus-mediated expression of human apolipoprotein E2 for preventative and therapeutic treatment of conditions associated with the amyloid β-peptide (Aβ), such as Alzheimer's disease, Down' syndrome, and cerebral amyloid angiopathy.

The Aβ peptide in circulating form is composed of 39-43 amino acids (mostly 40 or 42 amino acids) resulting from the cleavage of a precursor protein, amyloid precursor protein (APP). Conversion of Aβ from soluble to insoluble forms with high β-sheet content and its deposition as neuritic and cerebrovascular plaques in the brain appears to be associated with AD and other diseases, such as Down's syndrome, and pre-clinical and clinical cerebral amyloid angiopathy (CAA). Prevention and/or reduction of brain Aβ burden may therefore prevent, reduce, or reverse formation of neuritic plaques. Accordingly, methods of preventing and/or reducing brain Aβ burden may be therapeutically beneficial for conditions associated with brain Aβ burden.

A genetic association has been established between the risk to develop late-onset AD and apolipoprotein E (apoE) polymorphisms. ApoE is a 34-kDa very low-density lipoprotein which is primarily synthesized by the liver in association with the function of apoE in the periphery as a mediator of lipoprotein metabolism and lipid clearance. Astrocytes and microglia primarily synthesize apoE in the central nervous system, where apoE is thought to function in the redistribution of lipid and cholesterol during membrane repair and has been implicated as being important for maintaining synaptic plasticity.

In humans, apoE has three major isoforms, apoE2, apoE3, and apoE4, which respectively are encoded by the ε2, ε3, and ε4 allelic variants of the APOE gene. The frequency at which these alleles occur is approximately 78% for ε3, 15% for ε4, and 7% for ε2. The human major apoE isoforms differ by single amino acid substitutions at positions 112 and 158 in the apoE polypeptide, where apoE3 contains Cys 112, Arg 158, apoE4 contains Arg 112, Arg 158,and apoE2 contains Cys 112, Cys 158. Individuals carrying one or two ε4 alleles develop AD at a younger age and have higher brain amyloid burden compared to individuals carrying two ε3 alleles. Moreover, genetic epidemiological studies suggest a protective role for the ε2 allele, which reduces the risk of AD.

Both in vitro and in vivo studies suggest that apoE alters brain deposition and/or clearance of Aβ in an apoE isoform-dependent manner. ApoE2 thus may be therapeutically beneficial for treating conditions associated with the Aβ peptide through prevention and/or reversal of Aβ deposition. WO 97/16458 describes inhibition of progression or onset of AD by administration of recombinant ApoE2 to the brain of a patient with early onset AD or a patient genetically at risk of developing the disease. WO 00/23587 describes a method of providing an individual with a higher amyloid β processing capacity by treating glial-cell progenitor cells through APOE gene therapy and then providing the cells to the individual.

It remains to be seen whether these or other methods in the art demonstrate alteration of brain Aβ pathology or levels in response to treatment. Thus, there is a need in the art for additional methods of treating conditions associated with the Aβ peptide through prevention and/or reduction of Aβ brain burden and corresponding Aβ deposition.

### SUMMARY OF THE INVENTION

This invention provides a method of preventing and/or reducing brain Aβ burden in a subject in need thereof comprising administering to a target site of the brain of the subject an effective amount of an apoE2 lentiviral expression vector.

This invention provides a method of inhibiting a condition or disease associated with Aβ in a subject in need thereof comprising administering to a target site of the brain of the subject an effective amount of an apoE2 lentiviral expression vector.

The invention further includes a method of reducing progression of a condition or disease associated with Aβ in a subject in need thereof, comprising administering to a target site of the brain of the subject an effective amount of an apoE2 lentiviral expression vector.

### DETAILED DESCRIPTION OF THE INVENTION

The term “apoE” as used herein refers to apolipoprotein E, while “APOE” refers to the gene encoding apoE. In accordance with the art, the three major isoforms of human apoE are designated apoE2, apoE3, and apoE4.

As used herein, “apoE2 lentiviral expression vector” refers to a lentivirus expression vector which is capable of transducing neuronal cells and expressing apoE2 within those cells. As used throughout, a lentivirus expression vector that expresses a particular transgene generally may be referred to as “lentiviral X” or “lenti-X”, where “X” refers to the polypeptide expressed from the transgene. For example, an apoE2 lentiviral expression vector may be referred to as “lentiviral apoE2” or “lenti-apoE2.”

As used herein, “subject” refers to a mammal, preferably a human. A subject will benefit from the present invention if the subject has a condition or disease caused by or related to the presence of toxic forms of Aβ in the subject's brain.

Subjects that will benefit from the present invention include those that display symptoms of a condition or disease related to Aβ. In particular, subjects who are either heterozygous or homozygous for apoE4 and that display indications of a condition or disease related to Aβ will benefit from the present invention.

By “condition or disease related to Aβ” is meant conditions and diseases that are associated with: 1) the development of β-amyloid plaques in the brain, 2) the synthesis of abnormal forms of Aβ, 3) the formation of particularly toxic forms of Aβ, or 4) abnormal rates of synthesis, degradation, or clearance of Aβ. Conditions and diseases such as clinical and pre-clinical Alzheimer's disease, Down's syndrome, cerebral amyloid angiopathy, certain vascular dementias, and mild cognitive impairment are known or suspected of having relationship to AD. “Disease progression” refers to worsening of signs or symptoms of the condition or disease with time.

Alzheimer's disease is the most prevalent disease related to Aβ (60-80% of dementias). Definite diagnosis of AD is only possible presently with a post-mortem examination. But, a diagnosis of probable AD correlates highly with AD pathology. Vascular dementia (VaD), dementia with Lewy bodies (DLB), and frontotemporal dementia (FFD) together probably account for 15% to 20% of dementias, with other disorders (e.g., hydrocephalus; vitamin B12 deficiency) accounting for about 5%. Of these, only certain vascular dementias are suspected of having a significant Aβ component.

Even though the nature or concentration of Aβ in a subject's brain may not be known with certainty, specialized centers can diagnose AD correctly up to 90 percent of the time (Alzheimer's Disease Fact Sheet, National Institutes of Health (NIH) Publication No. 03-3431, 2003). Diagnosis of AD can be made from a combination of assessments which includes examining a subject's general health, past medical problems, and whether the subject has difficulties in carrying out daily activities; testing bodily fluids such as blood and urine; testing memory, problem solving, attention, counting, and language; and performing brain scans (Alzheimer's Disease Fact Sheet, NIH Publication No. 03-3431, 2003).

A state of increased risk or early manifestation of cognitive problems that often progresses to AD is termed mild cognitive impairment (MCI). MCI is a clinical entity characterized by memory loss, without significant dysfunction in other cognitive domains and without impairment in activities of daily living (ADL) function. Early diagnosis and treatment of MCI, including with the use of the present invention, is important. Currently the best predictor of preclinical AD is a diagnosis of MCI, because 30-50% of subjects with MCI develop AD within 3-5 years. One structural correlate of MCI that may be predictive for which subjects will develop AD is the volume of the hippocampus. Subjects with MCI have smaller hippocampi than age-equivalent controls and appear to experience atrophy of the structure at a more rapid pace.

By “administering” is meant the act of introducing a pharmaceutical agent into the subject's body. Direct intracerebral injection is the preferred route of administering the pharmaceutical agent, apoE2 lentiviral expression vector, in the methods of the present invention.

An “effective amount” as used herein refers to an amount of apoE2 lentiviral vector which when administered to the subject will cause inhibition or reduction in progression of an Aβ-related condition or disease.

The term “target site” refers to the specific site in the brain which will be targeted for administration of the apoE2 lentiviral expression vector.

For those subjects in need of treatment for AD and other diseases associated with Aβ peptide, suitable target tissue or sites in the brain will be selected for administration of the apoE2 lentiviral expression vectors of the present invention. AD is associated with neuritic plaques of which deposited Aβ peptide is a primary constituent. These plaques are found in brain tissue including the cortex, hippocampus, subiculum, dentate gyrus, and amygdala. Accordingly, any of these regions of the brain will be suitable target sites for administration of lentiviral apoE2. Preferably, the target site is the hippocampus.

Another suitable target for administration of the apoE2 lentiviral expression vectors is the cerebrospinal fluid (CSF), which in turn could serve to deliver the vectors to particular brain tissue(s) of interest. One method of delivery to CSF is through injection of apoE2 lentiviral expression vectors into the choroids plexus, which in turn would secrete the lentiviral apoE2 into the CSF. In another method, apoE2 expression vectors will be administered into the ventricles of the brain and will enter into the CSF. Another mode of administration is injection of apoE2 lentiviral vectors into the spinal column so that it contacts the CSF.

The methods of the present invention utilize recombinant expression vectors to express apoE2 in the brain of a human subject in need thereof. Appropriate expression vectors for use in the invention must be able to transfect the non-dividing cells of the adult brain and be expressed in those cells. Several viral vectors are currently known to have such capability, including DNA viruses such as adenovirus and adeno-associated virus, and RNA viruses such as HIV-based lentivirus, feline immunodeficiency virus, and equine immunodeficiency virus. Preferably, a lentivirus is used as the vector in the present invention.

Lentiviral vectors offer several advantages over other viral vectors for use in the present invention. One advantage is that lentiviral vectors have been shown to efficiently transduce cells of the central nervous system after direct injection. Lentiviral vectors also have been shown to stably express a foreign transgene without detectable pathogenesis from viral proteins (see Naldini et al., P.N.A.S. USA 93:11382-88, 1996; Dull et al., J. Virol. 72:8463-67, 1998; Miyoshi et al., J. Virol. 72:8150-57, 1998). Moreover, expression of the lentiviral transgene can theoretically be long term, even up to the life span of the host.

Suitable recombinant lentiviral expression vectors for use in the present invention can be constructed according to means known to one of skill in the art. For example, lentiviral vectors expressing apoE2 can be produced using a four plasmid transfection system, as previously described (Dull et al., J. Virol. 72:8463-67, 1998; Miyoshi et al., J. Virol. 72:8150-57, 1998). Preparation of a lentiviral vector encoding apoE2 is described in Example 1. The sequence of apoE2 is known in the art, and recombinant DNA encoding apoE2 can be readily determined by a skilled artisan using the genetic code.

The recombinant lentiviral expression vectors of the present invention may be administered as pharmaceutical compositions designed to be appropriate for intracerebral injection. These pharmaceutical compositions may include pharmaceutically acceptable excipients such as buffers, surfactants, preservatives, solubilizing agents, isotonicity agents, stabilizing agents and the like are used as appropriate. Remington's Pharmaceutical Sciences, Mack Publishing Co., Easton Pa., latest edition, incorporated herein by reference, provides a compendium of formulation techniques as are generally known to practitioners.

The formulation can be sterile filtered after making the formulation, or otherwise made microbiologically acceptable. Therapeutic agents of the invention can be frozen or lyophilized for storage and reconstituted in a suitable sterile carrier prior to use. Lyophilization and reconstitution can lead to varying degrees of lentiviral activity loss. Dosages may have to be adjusted to compensate. The pH of the formulation will be selected to balance antibody stability (chemical and physical) and comfort to the patient when administered. Generally, pH between 4 and 8 is tolerated.

Preferably very small volumes of lentivirus are used for transduction in brain tissue. As illustrated in Example 1, a lentiviral vector preparation is concentrated by ultracentrifugation. The resulting preparation should have between 1×10⁸ to 1×10¹⁰ transducing units/ml (TU/ml), and at least have 1×10⁸ TU/ml, more preferably at least 1×10⁹ TU/ml, and even more preferably at least 1×10¹⁰ TU/ml. For viral vectors, concentration is defined by the vector titer or number of transducing viral particles/ml, which is empirically determined and expressed as TU/ml. As shown in Example 1, an estimate of the amount of transducing units (TU) in a lentiviral preparation described therein can be determined by first measuring the amount of HIV p24 gag antigen. Next, the p24 amount is correlated to a biological titer of a vector expressing green fluorescent protein (GFP) under the cytomegalovirus (CMV) promoter, as measured by flow cytometry. Finally, a conversion factor or 100,000 TU per ng of p24 is used to estimate vector titers. As illustrated in Example 1, a viral preparation may be concentrated to the desired range of TU/ml by centrifugation.

Delivery of a lentiviral vector may be achieved by means familiar to those of skill in the art, including microinjection through a surgical incision (see, e.g., Capecchi, Cell, 22:479-488 (1980)); electroporation (see, e.g., Andreason and Evans, Biotechniques, 6:650-660 (1988)); infusion, chemical complexation with a targeting molecule or co-precipitant (e.g., liposome, calcium), and microparticle bombardment of the target tissue (Tang, et al., Nature, 356:152-154 (1992)). Preferably, delivery is made via microinjection. Injection may be made unilaterally to a target site in one hemisphere of the brain. Preferably, stereotaxic injection is used to deliver lentiviral vector to target sites in both hemispheres of the brain. Alternatively, an injection is used to deliver lentiviral vector to the CSF through injection of the choroids plexus, brain ventricles, or the spinal column.

In consideration of the need for injection of small volumes to the brain, a dosage of a pharmaceutical composition having a volume between 1-25 μl, more preferably 1-10 μl is delivered to a target site in the brain. Preferably, a pharmaceutical composition contains at least 1×10⁸ TU/ml lentiviral vector. The delivery is accomplished slowly, such as over a period of 5-10 minutes, depending on the total volume of lentiviral vector composition to be delivered.

Those of skill in the art will appreciate that the direct delivery method employed by the invention reduces the risk that in vivo use of gene therapy will result in transfection of non-targeted cells with the lentiviral vector. According to the invention, delivery is preferably direct and the delivery sites are chosen so expression of apoE2 takes place over a controlled and predetermined region of the brain to optimize contact with targeted neurons, while minimizing contact with non-targeted cells.

The following examples are intended to illustrate but not to limit the invention.

### EXAMPLE 1

### Lentiviral Vector Production

Vector plasmids were constructed for the production of lentiviral vectors that express each of the human apoE isoforms and green fluorescent protein (GFP). The human cytomegalovirus (CMV) promoter was used to drive expression of the transgene. All vectors were designed to be self-inactivating (SIN) (Miyoshi et al., J. Virol. 72:8150-57, 1998) and utilized the woodchuck hepatitis virus post-transcriptional regulatory element (WPRE) downstream from the transgene (Zufferey et al., J. Virol. 73:2886-92, 1999). The HIV-1 central poly-purine track (cppt) was also located upstream of the CMV promoter (Follenzi et al., Nat. Genet. 25:217-22, 2000).

Lentiviral vectors were produced using a four-plasmid transfection system, as previously described (Dull et al., J. Virol. 72:8463-71, 1998; Miyoshi et al., J. Virol. 72:8150-57, 1998). Briefly, two packaging plasmids (encoding HIV gag-pol, and rev) together with a plasmid coding for vesicular stomatitus virus G-protein (VSV-G) (Stitz et al., Virol. 273:16-20, 2000) and the vector plasmid itself were transfected into 293T cells using the calcium phosphate method (Chen and Okayama, Mol. Cell. Biol. 7:2745-52, 1987; Graham and van der Eb, Virol. 52:456-67, 1973). Typically, 12 to 24 15-cm dishes of cells were transfected and the virus was harvested by collecting the cell culture medium after changing the transfection medium to DMEM containing 10% FCS. After filtering the collected medium through 0.22 μm filters, the virus was concentrated by centrifugation at 68,000×g for 2 hours, followed by a second centrifugation of the pooled resuspension at 59,000×g for 2 hours at room temperature. The resulting pellet was resuspended in 200 to 400 μl of Hanks' buffer. The titers of lentiviral vector titers were determined by measuring the amount of HIV p24 gag antigen with an ELISA kit (Perkin Elmer Life Science, Boston, Mass.). To estimate the amount of transducing units (TU), the p24 titer was correlated to the biological titer of a vector expressing GFP under the CMV promoter, as measured by flow cytometry. A conversion factor of 100,000 TU per ng of p24 was used to estimate vector titers. Expression of apolipoprotein E from vector-transduced cells was confirmed by immunoblot of cell culture supernatants using an apolipoprotein E specific antibody (E-19, Santa Cruz, Santa Cruz, Calif.). Each of the three lenti-apoE vectors was effective in transfecting and expressing human apoE in human 293T cells.

### EXAMPLE 2

### Administration of Lentiviral ApoE to a Mouse Model of AD and Analyses Following Five Weeks Exposure

The transgenic APP^{V717F} mice, also termed PDAPP mice, used in this study overexpress a mutated form of human amyloid precursor protein (APP) under the control of the Platelet-Derived Growth Factor promoter (Games et al., Nature 373:523-527, 1995). Additionally, a cohort of PDAPP mice lacking apoE (Bales et al., Nat. Genet. 17:263-64, 1997) was used to investigate the expression pattern of human apoE after lenti-vectors delivery into the brain. All experiments were conducted in compliance with protocols approved by the Institutional Animal Care and Use Committee.

In a first series of experiments, lentiviral vectors expressing GFP, apoE2, apoE3 or apoE4 were administered into the hippocampus of 8-9 month-old PDAPP mice (APP^{V717F}, apoE^{+/+}) or 11-13 month-old PDAPP mice lacking apoE (APP^{V717F}, apoE^{-/-}). For that purpose, mice were anesthetized with avertin (0.023 ml/g of body weight) and placed on a stereotaxic apparatus (Stoelting, Ill.). Lentiviral preparations (4×10⁹ TU/ml) were injected bilaterally (2 μl/site) into the CA3 region of the hippocampus (−2.0 mm antero-posterior from bregma, ±2.3 mm medio-lateral from bregma, and 1.7 mm below dura) via a 10 μl Hamilton syringe. Mice were then individually housed and allowed to recover from surgery. Five weeks after injection, the mice were deeply anesthetized with avertin and transcardiacally perfused with heparinized saline. Brains were rapidly removed from the skull, one hemisphere was processed for histological analyses, the other hemisphere dissected and frozen on dry ice for biochemical analyses.

Tissue Preparation and Biochemical Analyses.

Hippocampi were processed using a 3-step extraction procedure; each step was followed by centrifugation at 10,000 rpm for 10 minutes at 4° C. The first step consisted of homogenizing samples in cold PBS with proteinase inhibitors (Complete™, Boehringer-Mannheim, IN). The second step consisted of re-suspension of the pellet in RIPA (50 mM Tris, 150 mM NaCl, 0.5% DOC, 1% NP40, 0.1% SDS and Complete™, pH 8.0). The third step consisted of re-suspension of the pellet in 5M guanidine-HCl and rocking the tubes for 2 hours at room temperature.

Aβ₁₋₄₀ and Aβ₁₋₄₂ were quantified in each pool using an ELISA. Briefly, the monoclonal antibodies 2G3 and 21F12 (10 μg/ml each) were used to capture Aβ peptides terminating at residues 40 and 42, respectively. Biotinylated monoclonal antibody 3D6 (0.5 μg/ml), which recognizes the Aβ₁₋₅ region of human Aβ, was used as the reporter antibody.

Expression of apoE was determined by western blotting. Briefly, proteins from RIPA extracts were size fractionated by 10% or 15% Tris-HCl SDS-PAGE (Criterion gel, Bio-Rad) and transferred onto PVDF membrane. To detect apoE, the membrane was immunoblotted using a biotinylated goat anti-human apoE antibody (0.02 μg/ml; Biodesign), followed by Neutravidin-HRP (1:200,000; Pierce) and reacted with west-femto SuperSignal (Pierce). Similarly, primary antibodies against APP (CT695, 0.05 μg/ml; Zymed Laboratories), GFAP (1;2000; Chemicon) and NF70 (1:1000; Chemicon), followed by appropriate HRP-conjugated secondary antibodies (1:100,000; Santa Cruz) were used.

Tissue Preparation and Histological Analyses

Brains were drop-fixed in 4% paraformaldehyde for 4 hours, then transferred to 20% sucrose for 24-48 hours and frozen in liquid nitrogen. Serial saggital 20-μm thick sections were cut at −18° C. in a cryostat and placed on Superfrost slides. Brain sections were inmunoreacted with one of the following antibodies: goat polyclonal anti-human apoE (Chemicon, 1:500), rabbit polyclonal anti-GFAβ (Chemicon, 1:1000), mouse monoclonal 3D6 (recognizes free amino-terminal region of Aβ, 1:500), rabbit polyclonal PAN/Aβ (QCB, 1:250) or mouse monoclonal 21F12 (recognizes specifically Aβₓ₋₄₂, 1:1,000). The specificity of the immunoreactivity was confirmed by lack of signal when the primary antibody was omitted. Analysis of immunoreactive deposits for Aβ were performed on a PC by using the public domain NIH IMAGE J program (http://rsb.info.nih.gov/nih-image) by defining a region of interest and setting a threshold to discriminate non-specific staining. The percentage of surface area covered by Aβ immunoreactivity was used to measure Aβ burden. The region analyzed was an area of the hippocampal formation comprising layers oriens, pyramidal, and stratum radiatum, and the dentate gyrus.

Lentivirus Mediated Expression of apoE in the Hippocampus

We first investigated whether proteins of interest can be efficiently expressed in the hippocampus after intrathecal lenti-vector delivery. Transgenic mice were administered the viral vectors bilaterally into the hippocampus and sacrificed for histological and biochemical analyses five weeks later. In PDAPP mice lacking mouse apoE (APP^{V717F}, apoE^{-/-}) and administered lenti-apoE vectors, immunostaining of brain sections using an antibody directed against apoE revealed a diffuse pattern of immunoreactivity in the hippocampus. Although apoE immunoreactivity was more prominent at the injection site, intense immunolabelling was surprisingly observed throughout the entire hippocampus, particularly in the hilus of the dentate gyrus and along the mossy fibers projecting to CA3. Diffuse apoE immunoreactivity was also observed in the CA1 region, though much weaker than in the hilus of the dentate. Specificity of the signal was confirmed by the lack of immunoreactivity in control brain sections (either omission of the primary antibody or immunostaining of brain sections from mice not administered lenti-apoE vectors). The diffuse patterns of apoE immunoreactivity suggest that apoE is mainly secreted from transfected cells, however some cell bodies within the hilus of the dentate gyrus was also be detected. Double staining for GFP and cellular markers such as GFAP or NeuN revealed that neurons are the primary cell type transfected by lenti vectors in the brain. Furthermore, hippocampal samples from mice administered lenti-GFP (negative control) or lenti-apoE vectors were also analyzed by western blotting along with recombinant proteins (positive control) and hippocampal samples from mice expressing more “physiological” levels of human apoE3 (apoE targeted-replacement mice, Sullivan et al., J. Biol. Chem. 272:17972-80, 1997). Immunoblotting revealed a 34 kD band corresponding to apoE in groups of mice administered lenti-apoE vectors. The amount of total protein loaded on the gel was slightly higher in samples from apoE targeted-replacement mice. Nonetheless apoE expression in mice administered lenti-apoE vectors surprisingly appeared to reach physiological levels.

Gene Delivery of Human apoE4 Increases Brain Aβ Burden in PDAPP Mice

We next examined whether expression of human apoE via the lenti-apoE vectors could alter brain Aβ burden in PDAPP mice lacking mouse apoE. In these mice, expression of lenti-apoE4 for 5 weeks resulted in a 2-3 fold increase in insoluble (guanidine extractable) Aβ1-42 in the hippocampus (p<0.05 versus GFP, apoE2 and apoE3), and a trend towards increased RIPA extractable Aβ1-42 (p<0.06 versus GFP, p<0.10 versus apoE2 and apoE3). Lenti-apoE4 treatment also resulted in a significant increase in PBS extractable Aβ1-42 (p<0.05 versus GFP, apoE2 and apoE3). Moreover, Aβ1-42 levels did not differ between groups administered lenti-GFP, lenti-apoE2 or lenti-apoE3. Hippocampal levels of Aβ1-40 were also slightly increased in lenti-apoE4 treated mice, but only in PBS-extractable (P<0.05 versus lenti-apoE2 and lenti-apoE3 groups) and RIPA-extractable (P<0.05 versus lenti-apoE3) fractions.

Hippocampal Aβ burden (percentage area covered by Aβ immunoreactivity) was also significantly increased in lenti-apoE4 treated mice compared to lenti-GFP and lenti-apoE3 treated mice (p<0.05, FIG. 4A). Also, the presence of amyloid deposits (Congo red positive) were observed in 80% of mice administered lenti-apoE4 compared to 0%, 33% and 11% of mice administered lenti-GFP, lenti-apoE2 or lenti-apoE3, respectively (Kruskal-Wallis test: p<0.01) (FIG. 4B). Only the lenti-apoE4 treated group was significantly different from the lenti-GFP treated group (Mann-Whitney test: p<0.01, FIG. 4B).

Lentiviral apoE2 Reduces Brain Aβ Burden in PDAPP Mice

We next investigated whether expression of human apoE via lenti-vectors delivery can alter brain Aβ pathology in PDAPP mice (APP^{V717F}, apoE^{+/+}). Levels of hippocampal Aβ were measured using a specific ELISA for human Aβ1-40 and Aβ1-42. Five weeks after lenti-vector delivery, a significant reduction of guanidine-extractable (insoluble) Aβ1-42 could be measured in the hippocampus of lenti-apoE2 treated mice (p<0.01 vs. lenti-apoE3 and lenti-apoE4, p=0.0533 vs. lenti-GFP) (Table 1). Hippocampal Aβ1-40 also tend to decrease in lenti-apoE2 mice, however this did not reach statistical significance (ANOVA group effect: F(3,23)=2.801, p=0.0626). By contrast, levels of PBS-extractable and RIPA-extractable Aβ peptides remained unchanged.

These ELISA results paralleled the results obtained from quantitative analyses of Aβ immunoreactivity on brain sections. Indeed, hippocampal Aβ burden differed between groups ](F3,23)=3.871, p<0.05] with lenti-apoE2 mice showing a significantly reduced Aβ burden compared to lenti-apoE3 and lenti-apoE4 mice (p<0.01 and p<0.05, respectively) and a trend compared to lenti-GFP mice (p=0.10) (Table 2).

TABLE 1
Levels of Aβ40 and Aβ42 in guanidine-extractable
hippocampal samples.

| | Aβ40 (ng/mg) | Aβ42 (ng/mg) |
|---|---|---|
| Lenti-GFP | 4.254 ± 1.025 | 4.289 ± 0.478 |
| Lenti-apoE2 | 2.627 ± 0.896 | 2.818 ± 0.717**# |
| Lenti-apoE3 | 6.002 ± 0.382 | 4.948 ± 0.290 |
| Lenti-apoE4 | 5.015 ± 1.075 | 5.056 ± 0.462 |

**p < 0.01 vs. lenti-apoE3 and lenti-apoE4 groups
#p = 0.0533 vs. lenti-GFP group

TABLE 2
Hippocampal Aβ burden (PAN/Aβ immunoreactivity)
in lentivector-treated PDAPP mice.

| | Aβ burden | SEM |
|---|---|---|
| Lenti-GFP | 4.908 | ±1.259 |
| Lenti-apoE2 | 1.662**# | ±1.276 |
| Lenti-apoE3 | 9.918 | ±1.627 |
| Lenti-apoE4 | 9.056 | ±2.828 |

**p < 0.01 vs. lenti-apoE3 and
#p < 0.05 vs. lenti-apoE4

### EXAMPLE 3

### Administration of Lentiviral ApoE to a Mouse Model of AD and Analyses Following Twelve Weeks Exposure

In a second series of experiments, three groups of 10 month-old PDAPP mice (APP^{V717F}, apoE^{+/+}) were administered lentiviral preparations (GFP, apoE2 or apoE4) as described in Example 2. A fourth group of age-matched PDAPP mice, which did not receive any treatment, was added as an alternative control. These mice were sacrificed 12 weeks after injection of lenti-vectors and analyzed as described in Example 2.

Similar results were observed for mice sacrificed 12 weeks after delivery of lenti-vectors compared to those mice sacrificed 5 weeks after delivery of lenti-vectors. In the mice exposed to lenti-vectors for 12 weeks, guanidine-extractable hippocampal Aβ1-42 levels and Aβ burden were decreased by 53% and 62% compared to controls, respectively (Tables 3 and 4). The decrease in Aβ1-42 did not reach statistical significance (lenti-GFP vs. lenti-apoE2, p=0.0583), however the decrease in Aβ burden was highly significant (lenti-GFP vs. lenti apoE2, p<0.001; lenti-apoE2 vs. lenti-apoE4, p<0.05). To confirm the latter result, the Aβ burden was further quantified on brain sections immunostained using a different antibody recognizing the free amino-terminal region of Aβ (mouse monoclonal 3D6). When using this antibody to stain Aβ deposits, we observed a highly significant decrease of hippocampal Aβ burden in both lenti-apoE2 and lenti-apoE4 mice compared to controls (p<0.002 and p<0.02, respectively) (Table 5).

TABLE 3
Levels of Aβ40 and Aβ42 in guanidine-extractable
hippocampal samples.

| | Aβ40 (ng/mg) | Aβ42 (ng/mg) |
|---|---|---|
| CTL | 982.3 ± 289.7 | 2053.2 ± 432.5 |
| Lenti-apoE2 | 323.6 ± 113.9 | 954.4 ± 288.1# |
| Lenti-apoE4 | 783.4 ± 180.4 | 1813.2 ± 342.6 |

#p = 0.0583 vs. CTL group

TABLE 4
Hippocampal Aβ burden (PAN/Aβ immunoreactivity)
in lentivector-treated PDAPP mice.

| | Aβ burden | SEM |
|---|---|---|
| CTL | 24.002 | ±2.703 |
| Lenti-apoE2 | 9.242***# | ±3.041 |
| Lenti-apoE4 | 18.558 | ±2.546 |

***p < 0.001 vs. CTL and
#p < 0.05 vs. lenti-apoE4

TABLE 5
Hippocampal Aβ burden (3D6 immunoreactivity) in
lentivector-treated PDAPP mice.

| | Aβ burden | SEM |
|---|---|---|
| CTL | 7.099 | 1.124 |
| Lenti-apoE2 | 2.39** | 0.807 |
| Lenti-apoE4 | 3.802* | 0.590 |

**p < 0.001 and
*p < 0.05 vs. CTL

### EXAMPLE 4

### Second Administration of Lentiviral ApoE to a Mouse Model of AD and Analyses Following Twelve Weeks Exposure

In a third series of experiments, a cohort of 7 month-old PDAPP mice were administered lenti-GFP, lenti-apoE2 or lenti-apoE4 into the CA1 region of the hippocampus and sacrificed 5 weeks later. Similar to the CA3 hippocampal region experiments described in Examples 2 and 3, good expression of GFP was observed following the CA1 administration of lenti-GFP. Consistent with our previous observation following administration into the CA3 region, PDAPP mice administered lenti-apoE2 into the CA1 region showed lower levels of hippocampal insoluble Aβ1-42 and reduced hippocampal Aβ burden compared to lenti-apoE4 mice (p<0.06 and p<0.07, respectively). Despite the age difference between mice utilized in our first study and in this study (9 month-old versus 7 month-old, respectively) and a difference between the injections sites (CA3 versus CA1, respectively), the two studies yielded very similar results. To compare results from both studies, we expressed the Aβ burden as a percentage of control (i.e. percentage hippocampal Aβ burden in lenti-apoE2 and lenti-apoE4 treated mice compared to lenti-GFP treated mice). When combining results from both studies, Aβ burden in lenti-apoE2 treated mice was dramatically reduced compared to both lenti-GFP treated mice (p<0.05) and lenti-apoE4 treated mice (p<0.01).

1. A method of inhibiting a condition or disease associated with Aβ in a subject in need thereof, comprising administering to a target site of the brain of the subject an effective amount of an apoE2 lentiviral expression vector.

2. The method of claim 1, wherein the subject is a human.

3. The method of claim 2, wherein the subject is genetically homozygous for APOE4.

4. The method of claim 2, wherein the subject is genetically heterozygous for APOE4.

5. The method of claim 2, wherein the target site of the brain is selected from cortex, hippocampus, subiculum, dentate gyrus, amygdala, and cerebrospinal fluid.

6. The method of claim 5, wherein the target site is hippocampus.

7. The method of claim 1, wherein the apoE2 lentiviral expression vector is administered by direct intracerebral injection.

8. The method of claim 7, wherein the apoE2 lentiviral expression vector is administered by direct stereotaxic intracerebral injection.

9. The method of claim 1 wherein the apoE2 lentiviral expression vector is present in a pharmaceutical composition at a concentration from 1×10⁸ to 1×10¹⁰ transducing units/ml.

10. The method of claim 9 wherein from 2 μl to 10 μl of the pharmaceutical composition is administered to the target site.

11. A method of reducing progression of a condition or disease associated with Aβ in a subject in need thereof, comprising administering to a target site of the brain of the subject an effective amount of an apoE2 lentiviral expression vector.

12. The method of claim 11, wherein the subject is a human.

13. The method of claim 12, wherein the subject is genetically homozygous for APOE4.

14. The method of claim 12, wherein the subject is genetically heterozygous for APOE4.

15. The method of claim 12, wherein the target site of the brain is selected from cortex, hippocampus, subiculum, dentate gyrus, amygdala, and cerebrospinal fluid.

16. The method of claim 15, wherein the target site is hippocampus.

17. The method of claim 11, wherein the apoE2 lentiviral expression vector is administered by direct intracerebral injection.

18. The method of claim 17, wherein the apoE2 lentiviral expression vector is administered by direct stereotaxic intracerebral injection.

19. The method of claim 11 wherein the apoE2 lentiviral expression vector is present in a pharmaceutical composition at a concentration of at least 1×10⁸ transducing units/ml.

20. The method of claim 11 wherein the apoE2 lentiviral expression vector is present in a pharmaceutical composition at a concentration from 1×10⁸ to 1×10¹⁰ transducing units/ml.

21. The method of claim 19 wherein from 2 μl to 10 μl of the pharmaceutical composition is administered to the target site.

22. The method of claim 1, wherein the condition or disease is selected from Alzheimer's disease, Down's syndrome, cerebral amyloid angiopathy, and mild cognitive impairment.

23. The method of claim 22, wherein the condition or disease is Alzheimer's disease.

24. The method of claim 22, wherein the condition or disease is Down's syndrome.

25. The method of claim 22, wherein the condition or disease is cerebral amyloid angiopathy.

26. The method of claim 22, wherein the condition or disease is mild cognitive impairment.

27. A method of preventing or reducing brain Aβ burden in a subject in need thereof, comprising administering to a target site of the brain of the subject an effective amount of an apoE2 lentiviral expression vector.

28. The method of claim 27, wherein the subject is a human.

29. The method of claim 28, wherein the subject is genetically homozygous for APOE4.

30. The method of claim 28, wherein the subject is genetically heterozygous for APOE4.

31. The method of claim 28, wherein the target site of the brain is selected from cortex, hippocampus, subiculum, dentate gyrus, amygdala, and cerebrospinal fluid.

32. The method of claim 31, wherein the target site is hippocampus.

33. The method of claim 27, wherein the apoE2 lentiviral expression vector is administered by direct intracerebral injection.

34. The method of claim 33, wherein the apoE2 lentiviral expression vector is administered by direct stereotaxic intracerebral injection.

35. The method of claim 27 wherein the apoE2 lentiviral expression vector is present in a pharmaceutical composition at a concentration from 1×10⁸ to 1×10¹⁰ transducing units/ml.

36. The method of claim 35 wherein from 2 μl to 10 μl of the pharmaceutical composition is administered to the target site.
